(19) United States
(12) Patent Application Publication
(10) Pub. No.: US 2026/0028569 A1
(43) Pub. Date: Jan. 29, 2026

(54) APPARATUS, SYSTEM, AND METHOD FOR STORING MEDICAL, PHARMACEUTICAL, OR BIOLOGICAL MEDIA

(71) Applicant: SAINT-GOBAIN PERFORMANCE PLASTICS CORPORATION, Malvern, PA (US)

(72) Inventors: James David BOGHOSIAN, Natick, MA (US); Katie L. Campbell, Northborough, MA (US); Nicolas Drolet, Chelmsford, MA (US); Jonathon W. Wheatley, Traverse City, MI (US); Darshan M.V., Kolar (IN); Elizabeth Grace Towle, Worcester, MA (US); Nicole M. Raisanen, Boston, MA (US); Christopher Carrozza, Washington, DC (US); Zachary Corriveau, Worcester, MA (US); Matt Erle Leinberger, Saginaw, MI (US)

(21) Appl. No.: 19/277,823

(22) Filed: Jul. 23, 2025

(30) Foreign Application Priority Data
Jul. 23, 2024 (IN) 202441056023

(51) Int. Cl. C12M 1/12; C12M 1/00; C12M 1/26

(52) U.S. Cl. CPC C12M 25/02; C12M 23/26; C12M 29/20; C12M 33/04

(57) ABSTRACT

An apparatus including: a core including an annular body defining an internal void oriented down a central axis having a top axial surface and a bottom axial surface; at least one retainer including an annular body adapted to couple to the top axial surface or the bottom axial surface of the core to form a circumferential engagement cavity defined radially between the at least one retainer and the core; at least one ring seal disposed within the engagement cavity; and at least one flexible film membrane partially disposed within the engagement cavity, where the at least one flexible film membrane at least partially encloses the internal void to provide for a medically, biologically, or pharmaceutically active environment within the internal void of the core.

### CROSS-REFERENCE TO RELATED APPLICATION(S)

This application claims priority to and the benefit of Indian Patent Application number 202441056023, entitled “APPARATUS, SYSTEM, AND METHOD FOR STORING MEDICAL, PHARMACEUTICAL, OR BIOLOGICAL MEDIA,” by James David BOGHOSIAN et al., filed Jul. 23, 2024, which is assigned to the current assignee hereof and incorporated herein by reference in its entirety.

### FIELD OF THE DISCLOSURE

The present disclosure relates to apparatuses, systems, and methods for storing medical, pharmaceutical, or biological media.

### RELATED ART

Many pharmaceutical and bioprocessing processes utilize storage containers for storing fluid associated with medical, pharmaceutical, or biological media such as, but not limited to, cell cultures. In certain instances, the container can include a bag having at least one flexible portion—such as a flexible sidewall. These flexible sidewalls may be challenging to manufacture and are prone to wrinkling, preventing efficient storage and lessening the desired modification or sustainment of the medical, pharmaceutical, or biological media within the container. Further, these flexible sidewalls may at least partially serve as membranes for gas diffusion into the container, which may also be lessened in efficiency by wrinkling. Therefore, the medical, pharmaceutical, and bioprocessing industries continue to demand better containers and associated technologies to permit more efficient storage and operation involving modification or sustainment of medical, pharmaceutical, and biological media.

### BRIEF DESCRIPTION OF THE DRAWINGS

Embodiments are illustrated by way of example and are not intended to be limited in the accompanying FIGURES.

FIG. 1A includes a side perspective view of an apparatus in accordance with an embodiment.

FIG. 1B includes a side perspective view of an apparatus in accordance with an embodiment.

FIG. 1C includes a cross-sectional view of an apparatus in accordance with an embodiment.

FIG. 1D includes a close-up cross-sectional view of an apparatus as viewed within circle D of FIG. 1C in accordance with an embodiment.

FIG. 1E1 includes a top view of a core for an apparatus in accordance with an embodiment.

FIG. 1E2 includes a side view of a core for an apparatus in accordance with an embodiment.

FIG. 1E3 includes an isometric view of a core for an apparatus in accordance with an embodiment.

FIG. 1F1 includes a top view of a retainer for an apparatus in accordance with an embodiment.

FIG. 1F2 includes a side view of a retainer for an apparatus in accordance with an embodiment.

FIG. 1F3 includes an isometric view of a retainer for an apparatus in accordance with an embodiment.

FIG. 1G1 includes a top view of a membrane for an apparatus in accordance with an embodiment.

FIG. 1G2 includes an isometric view of a membrane for an apparatus in accordance with an embodiment.

### DETAILED DESCRIPTION

The following description in combination with the FIGURES is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other embodiments can be used based on the teachings as disclosed in this application.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item may be used in place of a single item. Similarly, where more than one item is described herein, a single item may be substituted for that more than one item.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the medical, pharmaceutical, and biological media processing and storage arts.

In accordance with one or more of the embodiments described herein, an apparatus may generally include: a core including an annular body defining an internal void oriented down a central axis having a top axial surface and a bottom axial surface; at least one retainer including an annular body adapted to couple to the top axial surface or the bottom axial surface of the core to form a circumferential engagement cavity defined radially between the at least one retainer and the core; at least one ring seal disposed within the engagement cavity; and at least one flexible film membrane partially disposed within the engagement cavity, where the at least one flexible film membrane at least partially encloses the internal void to provide for a medically, biologically, or pharmaceutically active environment within the internal void of the core.

In accordance with one or more of the embodiments described herein, a system may generally include: a medium including a medical, pharmaceutical, or biological component; and an apparatus including: a core including an annular body defining an internal void oriented down a central axis having a top axial surface and a bottom axial surface, at least one retainer including an annular body adapted to couple to the top axial surface or the bottom axial surface of the core to form a circumferential engagement cavity defined radially between the at least one retainer and the core, at least one ring seal disposed within the engagement cavity, and at least one flexible film membrane partially disposed within the engagement cavity, where the at least one flexible film membrane at least partially encloses the internal void about the medium to provide for a medically, biologically, or pharmaceutically active environment within the internal void of the core.

In accordance with one or more of the embodiments described herein, a method may generally include: providing a medium including a medical, pharmaceutical, or biological component; providing an apparatus including: a core including an annular body defining an internal void oriented down a central axis having a top axial surface and a bottom axial surface, at least one retainer including an annular body adapted to couple to the top axial surface or the bottom axial surface of the core to form a circumferential engagement cavity defined radially between the at least one retainer and the core, at least one ring seal disposed within the engagement cavity, and at least one flexible film membrane partially disposed within the engagement cavity, where the at least one flexible film membrane at least partially encloses the internal void; and disposing the medium within the internal void to provide for a medically, biologically, or pharmaceutically active environment within the internal void of the core.

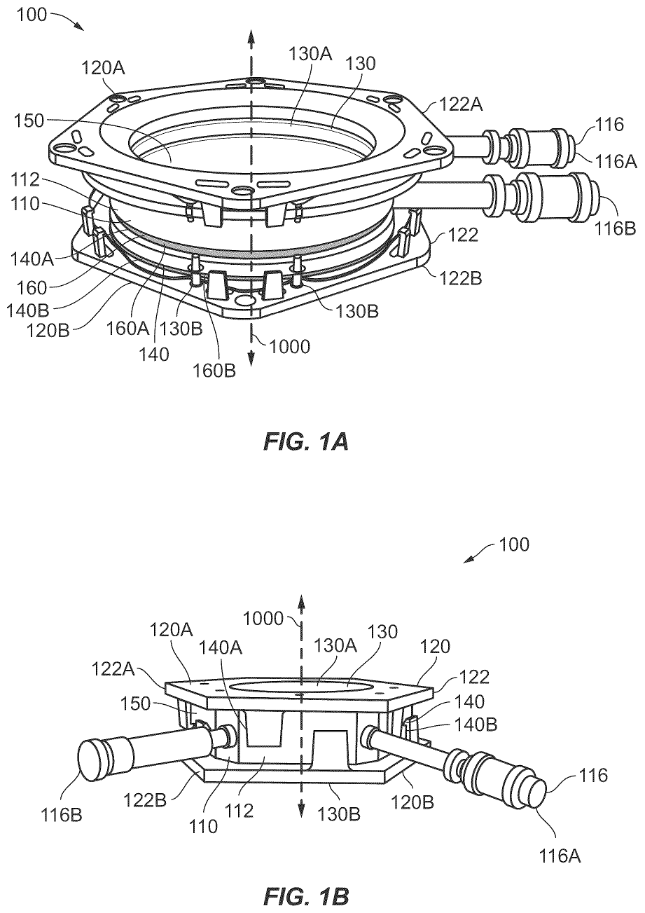

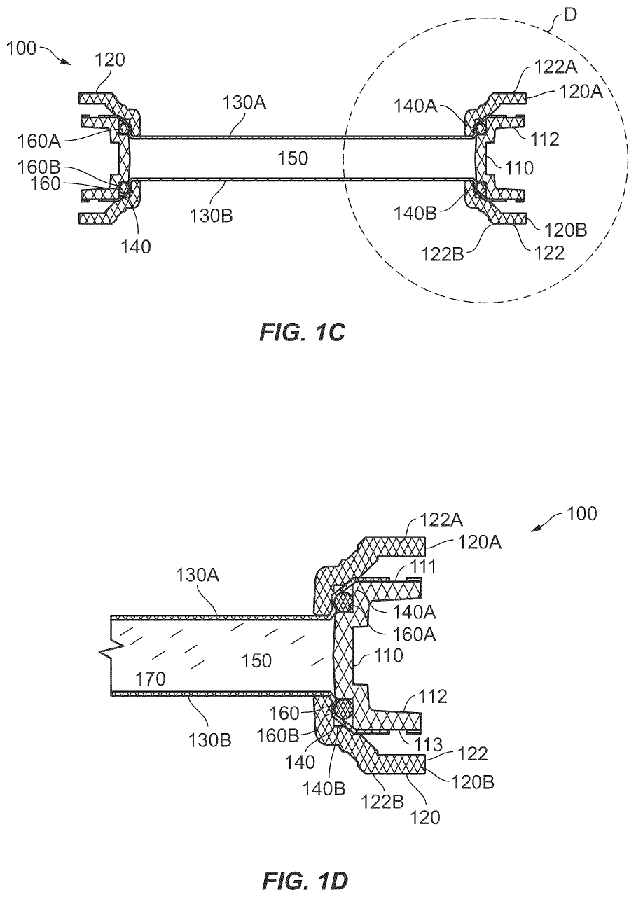

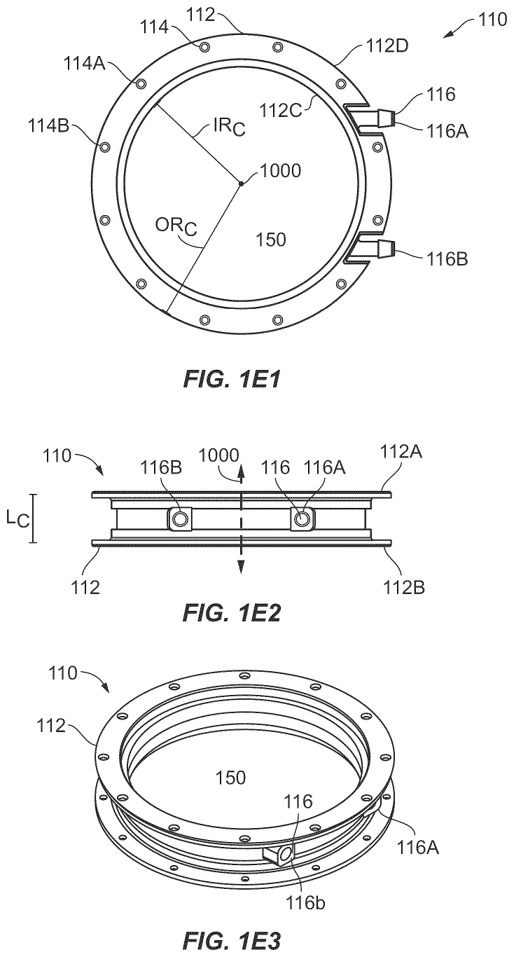

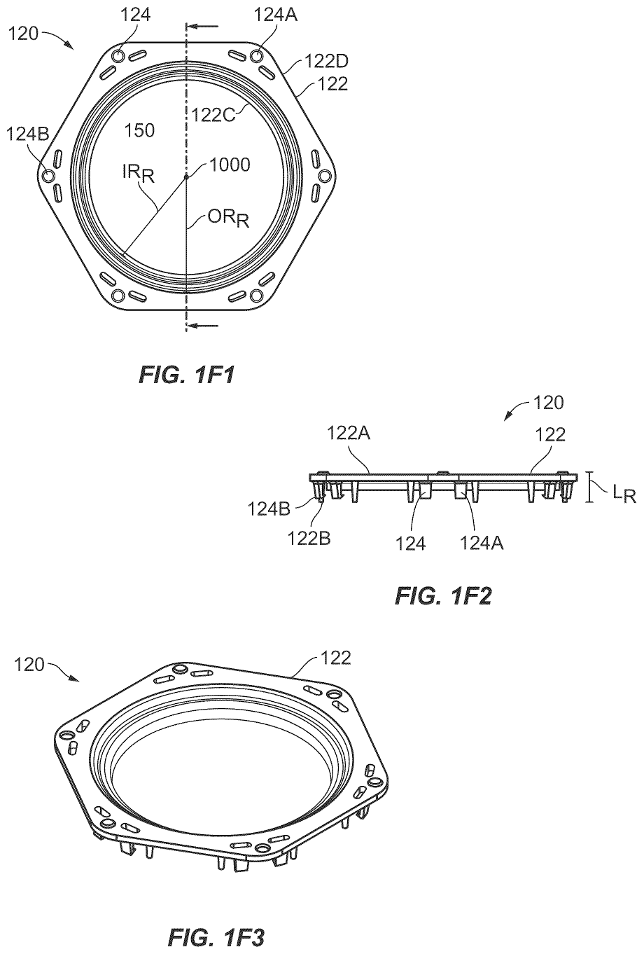

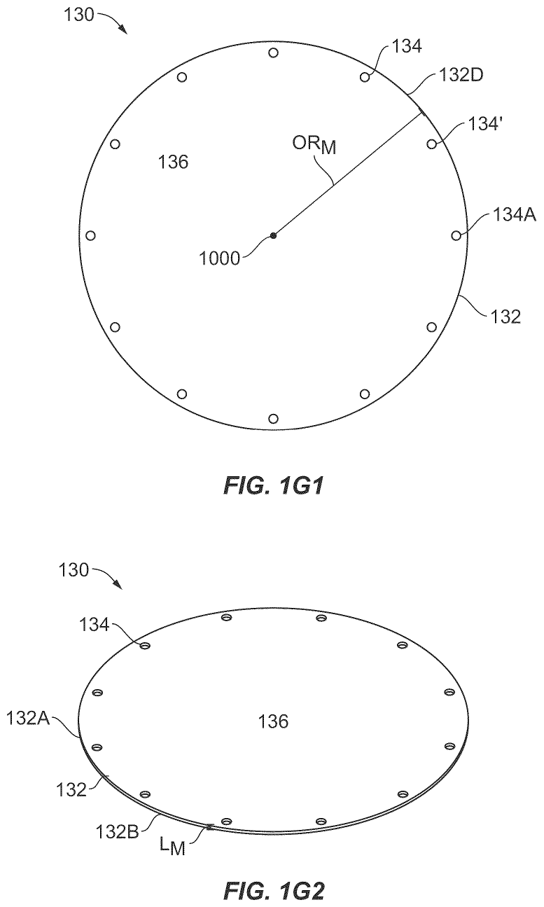

FIG. 1A includes a side perspective view of an apparatus 100 in accordance with an embodiment. FIG. 1B includes a side perspective view of an apparatus 100 in accordance with an embodiment. FIG. 1C includes a cross-sectional view of an apparatus in accordance with an embodiment. FIG. 1D includes a close-up cross-sectional view of an apparatus as viewed within circle D of FIG. 1C in accordance with an embodiment. It is contemplated that reference numerals are corresponding across all FIGURES disclosed herein. As shown in FIGS. 1A-1D, the apparatus 100 can generally include a core 110 oriented down a central axis 1000. In a number of embodiments, the core 110 may include an annular body 112. As used herein, the term “annular” may be defined as having a solid structure oriented 360° about the central axis 1000. The term “annular” including any cross-sectional shape (e.g. polygonal, oval, circular, semi-circular, or substantially circular cross-section) is contemplated herein. The annular body 112 of the core 110 may define an internal void 150 within an aperture of the annular body 112 down the central axis 1000. Further, as shown best in FIG. 1D, the annular body 112 of the core 110 may define a top axial surface 111 and a bottom axial surface 113 down the central axis 1000.

Further, still referring to FIGS. 1A-1D, the apparatus 100 can generally include at least one retainer 120 oriented down a central axis 1000. In a number of embodiments, as shown, the at least one retainer 120 may be adapted to couple to the top axial surface 111 or the bottom axial surface 113 of the annular body 112 of the core 110 down the central axis 1000. In a number of embodiments, the at least one retainer 120 may include an annular body 122. As shown in FIGS. 1A-1D, the apparatus 100 can generally include a plurality of retainers 120A, 120B including a plurality of annular bodies 122A, 122B. In a number of embodiments, the plurality of retainers 120A, 120B may be adapted to couple to the top axial surface 111 and the bottom axial surface 113 of the annular body 112 of the core 110 down the central axis 1000 (one on each axial side respectively). As shown best in FIGS. 1C-1D, as shown best in FIGS. 1C-1D, the at least one retainer 120 may be adapted to couple to the top axial surface 111 or the bottom axial surface 113 of the annular body 112 of the core 110 to form a circumferential engagement cavity 140 around a perimeter of the core 100. In a number of embodiments, as shown best in FIGS. 1C-1D, the at least one circumferential engagement cavity 140 may be formed and defined radially between the at least one retainer 120 and the core 110 such that the core 110 forms the outer diameter of the at least one circumferential engagement cavity 140 while the at least one retainer 120 forms the inner diameter of the at least one circumferential engagement cavity 140 relative to the central axis 1000. In a number of embodiments, as shown, the plurality of retainers 120A, 120B may be adapted to couple to the top axial surface 111 and the bottom axial surface 113 of the annular body 112 of the core 110 to form a plurality of circumferential engagement cavities 140A, 140B around a perimeter of the core 100.

Further, as shown best in FIGS. 1A and 1C-1D, the apparatus can generally include at least one ring seal 160 disposed within the engagement cavity 140. As shown in FIGS. 1A and 1C-1D, the apparatus 100 can generally include a plurality of ring seals 160A, 160B, one disposed in each engagement cavity 140A, 140B. The at least one ring seal 160 may have a polygonal, circular, elliptical, cross-sectional shape or may be another type. In a number of embodiments, the at least one ring seal 160 may include an elastomer material.

Further, the apparatus 100 can generally include at least one membrane 130 oriented down a central axis 1000 and at least partially disposed within the engagement cavity 140. As shown in FIGS. 1A-1D, the apparatus 100 can generally include a plurality of membranes 130A, 130B, one at least partially disposed in each engagement cavity 140A, 140B. Further, as shown in FIGS. 1A-1D the at least one membrane 130 may at least partially enclose the internal void 150 to provide for a medically, biologically, or pharmaceutically active environment within the internal void 150 of the core 110 and subsequently the apparatus 100. Further, as shown in FIGS. 1A-1D, the plurality of membranes 130A, 130B may together at least partially enclose the internal void 150 completely in the axial direction down the central axis 1000 to provide for a medically, biologically, or pharmaceutically active environment within the internal void 150 of the core 110 and subsequently the apparatus 100.

FIG. 1E1 includes a top view of a core for an apparatus in accordance with an embodiment. FIG. 1E2 includes a side view of a core for an apparatus in accordance with an embodiment. FIG. 1E3 includes an isometric view of a core for an apparatus in accordance with an embodiment. As shown in FIGS. 1E1-1E3, the core 110 may include an annular body 122 forming an “annular shape” surrounding a bore or aperture defining an internal void 150 down a central axis 1000. In particular embodiments, the core 110 may form a substantially circular shape, as shown. The body 112 of the core 110 may have a first axial end 112A, a second axial end 112B, an inner radial end 112C and an outer radial end 112D. As shown best in FIG. 1E2, in a number of embodiments, the core 100 may include a radial flange at at least one of the first axial end 112A or the second axial end 112B. As shown, in a number of embodiments, the core 100 may include a radial flange at both of the first axial end 112A and the second axial end 112B forming a substantially U-shaped cross-sectional profile in a plane parallel to the central axis 1000.

In a number of embodiments, as shown best in FIG. 1E1, the core 110 may have an outer radius OR_{C}. For purposes of embodiments described herein, the core 110 may have an outer radius OR_{C} is the distance from the central axis 1000 to the outer radial end 112D. According to certain embodiments, core 110 may have an outer radius OR_{C} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, core 110 may have an outer radius OR_{C} that may be not greater than about 1500 mm, not greater than about 1200 mm, or even not greater than about 1000 mm. It will be appreciated that the core 110 may have an outer radius OR_{C} that may be within a range between any of the minimum and maximum values noted above. It will be further appreciated that the core 110 may have an outer radius OR_{C} that may be any value between any of the minimum and maximum values noted above.

In a number of embodiments, as shown best in FIG. 1E1, the core 110 may have an inner radius IR_{C}. For purposes of embodiments described herein, the core 110 may have an inner radius IR_{C} is the distance from the central axis 1000 to the inner radial end 112C. According to certain embodiments, core 110 may have an inner radius IR_{C} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, core 110 may have an inner radius IR_{C} that may be not greater than about 1500 mm, not greater than about 1200 mm, or even not greater than about 1000 mm. It will be appreciated that the core 110 may have an inner radius IR_{C} that may be within a range between any of the minimum and maximum values noted above. It will be further appreciated that the core 110 may have an inner radius IR_{C} that may be any value between any of the minimum and maximum values noted above.

In a number of embodiments, as shown best in FIG. 1E2, the core 110 can have an axial length (or thickness) Lc. For purposes of embodiments described herein, the length Le of the core 110 is the distance from the first axial end 112A to the second axial end 112B. According to certain embodiments, the length Le of the core 110 may be at least about 5 mm, at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the length Le of the core 110 may be not greater than about 1500 mm, not greater than about 1200 mm, or even not greater than about 1000 mm. It will be appreciated that the length Lc of the core 110 may be within a range between any of the minimum and maximum values noted above. It will be further appreciated that the length Le of the core 110 may be any value between any of the minimum and maximum values noted above.

In a number of embodiments, as shown best in FIG. 1E1, the core 110 may include at least one axially oriented coupling component 114. In a number of embodiments, the at least one axially oriented coupling component 114 of the core 110 may couple to a neighboring retainer 120 within the apparatus. In a number of embodiments, as shown best in FIG. 1E1, the core 110 may include a plurality of axially oriented coupling components 114A, 114B. In a number of embodiments, the at least one axially oriented coupling component 114 may include at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, bores, welds, or combinations thereof. In particular embodiments, as shown in FIG. 1E1, the at least one axially oriented coupling component 114 may include a bore or groove.

In a number of embodiments, as shown best in FIGS. 1E1-1E3, the core 110 may include at least one access port 116 allowing for entrance or exit of a medium into or out of the apparatus 100. In a number of embodiments, as shown best in FIGS. 1E1-1E3, the core 110 may include at a plurality of access ports 116A, 116B allowing for entrance or exit of a medium into or out of the apparatus 100. In particular embodiments, as shown in FIG. 1E1-1E3, the at least one access port 116 may include a bore. In a number of embodiments, the at least one access port 116 may be adapted to permit, for example, sampling, transport, draining, or removal of medium from the apparatus 100. In a particular embodiment, at least one of the one or more access ports 116 can include at least one fluid port 11, at least two access ports 116, at least three access ports 116, at least four access ports 116, or at least five access ports 116. In another embodiment, the apparatus 100 can include no greater than twenty access ports 116, no greater than fifteen access ports 116, or no greater than ten access ports 116. In the illustrated embodiment, the access ports 116 may have a same shape and size. In other embodiments, the access ports 116 can have different shapes, sizes, lumen diameters, operational capacities, or any combination thereof relative to one another. In an embodiment, the one or more access ports 116 can be coupled with fluid lines adapted to permit transport of medium from the apparatus 100 to an equipment or other medium destination.

FIG. 1F1 includes a top view of a retainer for an apparatus in accordance with an embodiment. FIG. 1F2 includes a side view of a retainer for an apparatus in accordance with an embodiment. FIG. 1F3 includes an isometric view of a retainer for an apparatus in accordance with an embodiment. As shown in FIGS. 1F1-1F3, the at least one retainer 120 may include an annular body 122 forming an “annular shape” surrounding a bore or aperture defining an internal void 150 corresponding to the internal void of the apparatus 100 down a central axis 1000. In particular embodiments, the at least one retainer 120 may form a substantially hexagonal shape, as shown. The body 122 of the at least one retainer 120 may have a first axial end 122A, a second axial end 122B, an inner radial end 122C and an outer radial end 122D. As shown best in FIG. 1F2, in a number of embodiments, the at least one retainer 120 may include a radial flange at at least one of the first axial end 112A or the second axial end 112B. As shown, in a number of embodiments, the at least one retainer 120 may include a radial flange at both of the first axial end 122A and the second axial end 122B (the radial flange at the second end being a axially oriented coupling component as explained below) forming a substantially L-shaped cross-sectional profile in a plane parallel to the central axis 1000.

In a number of embodiments, as shown best in FIG. 1F1, the at least one retainer 120 may have an outer radius OR_{R}. For purposes of embodiments described herein, the at least one retainer 120 may have an outer radius OR_{R} is the distance from the central axis 1000 to the outer radial end 122D. According to certain embodiment, the at least one retainer 120 may have an outer radius OR_{R} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the at least one retainer 120 may have an outer radius OR_{R} that may be not greater than about 1500 mm, not greater than about 1200 mm, or even not greater than about 1000 mm. It will be appreciated that the at least one retainer 120 may have an outer radius OR_{R} that may be within a range between any of the minimum and maximum values noted above. It will be further appreciated that the at least one retainer 120 may have an outer radius OR_{R} that may be any value between any of the minimum and maximum values noted above.

In a number of embodiments, as shown best in FIG. 1F1, the at least one retainer 120 may have an inner radius IR_{R}. For purposes of embodiments described herein, the at least one retainer 120 may have an inner radius IR_{R} is the distance from the central axis 1000 to the inner radial end 122C. According to certain embodiment, the at least one retainer 120 may have an inner radius IR_{R} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the at least one retainer 120 may have an inner radius IR_{R} that may be not greater than about 1500 mm, not greater than about 1200 mm, or even not greater than about 1000 mm. It will be appreciated that the at least one retainer 120 may have an inner radius IR_{R} that may be within a range between any of the minimum and maximum values noted above. It will be further appreciated that the at least one retainer 120 may have an inner radius IR_{R} that may be any value between any of the minimum and maximum values noted above.

In a number of embodiments, as shown best in FIG. 1F2, the at least one retainer 120 can have an axial length (or thickness) LR. For purposes of embodiments described herein, the length LR of the at least one retainer 120 is the distance from the first axial end 112A to the second axial end 112B. According to certain embodiment, the length LR of the at least one retainer 120 may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the length LR of the at least one retainer 120 may be not greater than about 1500 mm, not greater than about 1200 mm, or even not greater than about 1000 mm. It will be appreciated that the length LR of the at least one retainer 120 may be within a range between any of the minimum and maximum values noted above. It will be further appreciated that the length LR of the at least one retainer 120 may be any value between any of the minimum and maximum values noted above.

In a number of embodiments, as shown best in FIG. 1F1, the at least one retainer 120 may include at least one axially oriented coupling component 124. In a number of embodiments, the at least one axially oriented coupling component 124 of the at least one retainer 120 may couple to a core 110 within the apparatus. In a number of embodiments, the at least one axially oriented coupling component 124 of the at least one retainer 120 may couple to a neighboring retainer 120 of a neighboring apparatus 100. In a number of embodiments, as shown best in FIG. 1F2, the at least one retainer 120 may include a plurality of axially oriented coupling components 124A, 124B. In a number of embodiments, the at least one axially oriented coupling component 124 may include at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, projection, bores, welds, or combinations thereof. In particular embodiments, as shown in FIG. 1F2, the at least one axially oriented coupling component 124 may include a tongue adapted to pair with a groove or bore of the core 100 to snap-fit the core and the retainer together. In particular embodiments, as shown in FIG. 1F1, the at least one axially oriented coupling component 124 may include a projection or groove to pair the at least one retainer 120 to a neighboring membrane 130 of a neighboring apparatus 100.

FIG. 1G1 includes a top view of a membrane for an apparatus in accordance with an embodiment. FIG. 1G2 includes an isometric view of a membrane for an apparatus in accordance with an embodiment. As shown in FIGS. 1G1-1G2, the at least one membrane 130 may include an annular body 132 forming an “annular shape” to be placed over the internal void 150 corresponding to the internal void of the apparatus 100 down a central axis 1000 to form a medically, biologically, or pharmaceutically active environment. In particular embodiments, the at least one membrane 130 may form a substantially circular shape with an outer radial end 132D, a first axial end 132A and a second axial end 132B, as shown and defining a major surface 136. In an embodiment, the at least one membrane 130 can include a media or particulate for filtering. In another embodiment, the at least one membrane 130 can include a porous structure such as a block or structure of material having perforations or micro-perforations. In a further embodiment, the at least one membrane 130 can include a mesh, a screen, or a woven or non-woven medium. In yet another embodiment, the at least one membrane 130 can include a combination of filter elements. In a number of embodiments, the at least one membrane 130 may include a polymer that is gas-permeable and liquid-impermeable. In a number of embodiments, the at least one membrane 130 may include a polymer including EVA, silicone, polyolefin, polycarbonate, HDPE, POE, COC, COP, PMP, FEP, PTFE, textured films (e.g. textured silicone, textured FEP, textured laminate), or a combination thereof. In a number of embodiments, the at least one membrane 130 may be a monolayer. In a number of embodiments, the at least one membrane 130 may be a multi-layer laminate.

In a number of embodiments, as shown best in FIG. 1G1, the at least one membrane 130 may have a radius OR_{M}. For purposes of embodiments described herein, the at least one membrane 130 may have an outer radius OR_{M} is the distance from the central axis 1000 to the outer radial end 132D. According to certain embodiment, the at least one membrane 130 may have an outer radius OR_{M} that may be at least about 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the at least one membrane 130 may have an outer radius OR_{M} that may be not greater than about 1500 mm, not greater than about 1200 mm, or even not greater than about 1000 mm. It will be appreciated that the at least one membrane 130 may have an outer radius OR_{M} that may be within a range between any of the minimum and maximum values noted above. It will be further appreciated that the at least one membrane 130 may have an outer radius OR_{M} that may be any value between any of the minimum and maximum values noted above.

In a number of embodiments, as shown best in FIG. 1G2, the at least one membrane 130 can have an axial length (or thickness) L_{M}. For purposes of embodiments described herein, the length L_{M} of the at least one membrane 130 is the distance from the first axial end 132A to the second axial end 132B. According to certain embodiment, the length L_{M} of the at least one membrane 130 may be at least about 0.0001 mm, such as 0.001 mm, such as 0.01 mm, such as 1 mm, such as 10 mm, at least 25 mm, at least 50 mm, at least 100 mm, at least about 150 mm, or at least about 200 mm, or at least about 250 mm, or at least about 300 mm, or even at least about 500 mm. According to still other embodiments, the length L_{M} of the at least one membrane 130 may be not greater than about 1500 mm, not greater than about 1200 mm, or even not greater than about 1000 mm. It will be appreciated that the length L_{M} of the at least one membrane 130 may be within a range between any of the minimum and maximum values noted above. It will be further appreciated that the length L_{M} of the at least one membrane 130 may be any value between any of the minimum and maximum values noted above.

In a number of embodiments, as shown best in FIG. 1G1, the at least one membrane 130 may include at least one axially oriented coupling component 134. In a number of embodiments, the at least one axially oriented coupling component 134 of the at least one membrane 130 may couple to a core 110 and/or retainer 120 within the apparatus. In a number of embodiments, as shown best in FIG. 1G2, the at least one membrane 130 may include a plurality of axially oriented coupling components 134A, 134B. In a number of embodiments, the at least one axially oriented coupling component 134 may include at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, projection, bores, welds, or combinations thereof. In particular embodiments, as shown in FIG. 1G2, the at least one axially oriented coupling component 134 may include a bore adapted to pair with a groove or bore of the core 100 to snap-fit the core and the membrane together with the membrane contained. In a further embodiment, the at least one membrane 130 can be adhered to at least one of the retainer 120 or the core 110 by an adhesive or compound. In a number of embodiments, referring back to FIGS. 1C-1D, the at least one retainer 120 may be adapted to couple to the top axial surface 111 or the bottom axial surface 113 of the annular body 112 of the core 110 to form a circumferential engagement cavity 140 which may be formed and defined radially between the at least one retainer 120 and the core 110 such that the core 110 forms the outer diameter of the at least one circumferential engagement cavity 140 while the at least one retainer 120 forms the inner diameter of the at least one circumferential engagement cavity 140 relative to the central axis 1000. In a number of embodiments, as shown, the apparatus can generally include at least one ring seal 160 and at least a part of a membrane 130 disposed within the engagement cavity 140. As shown in 1C-1D, the unique architecture of the circumferential engagement cavity 140 may allow the membrane 130 to be tensioned through the interaction of the at least one retainer 120, the core 110, and the ring seal 160 within the engagement cavity 140 to at least partially enclose the internal void 150. As shown in 1C-1D, the unique architecture of the circumferential engagement cavity 140 may allow the membrane 130 to be tensioned in a uniform circumferential pattern through the interaction of the at least one retainer 120, the core 110, and the ring seal 160 within the engagement cavity 140 to at least partially enclose the internal void 150.

In a number of embodiments, the at least one membrane 130 may be tensioned within an engagement cavity 140 such that it has a flatness against a line or plane perpendicular to the central axis 1000 of the apparatus. In a number of embodiments, the at least one membrane 130 may be tensioned within an engagement cavity 140 to have a deviation of at least about 0.0001 mm, such as 0.001 mm, such as 0.01 mm, such as 1 mm, such as 10 mm, at least 25 mm, at least 50 mm, at least 100 mm, at least about 150 mm, or at least about 200 mm, or at least about 250 mm, or at least about 300 mm, or even at least about 500 mm from a line or plane perpendicular to the central axis 1000 of the apparatus. According to still other embodiments, the at least one membrane 130 may be tensioned within an engagement cavity 140 to have a deviation that may be not greater than about 1500 mm, not greater than about 1200 mm, or even not greater than about 1000 mm a line or plane perpendicular to the central axis 1000 of the apparatus. It will be appreciated that the at least one membrane 130 may be tensioned within an engagement cavity 140 to have a deviation that may be within a range between any of the minimum and maximum values noted above. It will be further appreciated that the at least one membrane 130 may be tensioned within an engagement cavity 140 to have a deviation that may be any value between any of the minimum and maximum values noted above.

In a number of embodiments, the apparatus 100 or any components thereof listed herein (including, but not limited to, the core 110, the at least one retainer 120, the at least one membrane 130, or the ring seal 160) can include a single-piece body. In another embodiment, the apparatus 100 or any components thereof listed herein (including, but not limited to, the core 110, the at least one retainer 120, the at least one membrane 130, or the ring seal 160) can include a multi-piece construction. In a number of embodiments, the apparatus 100 or any components thereof listed herein (including, but not limited to, the core 110, the at least one retainer 120, the at least one membrane 130, or the ring seal 160) can include a single use apparatus. In another embodiment, the apparatus 100 or any components thereof listed herein (including, but not limited to, the core 110, the at least one retainer 120, the at least one membrane 130, or the ring seal 160) can be reusable.

The apparatus 100 or any components thereof listed herein (including, but not limited to, the core 110, the at least one retainer 120, the at least one membrane 130, or the ring seal 160) can be formed from any suitable material in the sealing arts. In a particular embodiment, the apparatus 100 or any components thereof listed herein can at least partially include a polymer. The polymer may be a thermoplastic or thermosetting polymer. The polymer may be selected from the group including a polyketone, a polyaramid, a polyphenylene sulfide, a polyethersulfone, a polyphenylene sulfone, a polyamideimide, ultra high molecular weight polyethylene, a fluoropolymer, a polybenzimidazole, a polyacetal, polybutylene terephthalate (PBT), polypropylene (PP), rubber modified polypropylene, (EPDM/PP), polycarbonate (PC), Acrylonitrile butadiene styrene (ABS), polyethylene terephthalate (PET), a polyimide (PI), polyetherimide, polyetheretherketone (PEEK), polyatheretherketone (PAEK), polyethylene (PE), a polysulfone, a polyamide (PA), thermoplastic polyurethane (TPU), polyphenylene oxide, polyphenylene sulfide (PPS), a polyurethane, a polyester, a liquid crystal polymer (LCP), an elastomer, or any combination thereof. In an embodiment, the apparatus 100 or any components thereof listed herein may include, or even consist essentially of, a fluoropolymer. Exemplary fluoropolymers include a polytetrafluoroethylene (PTFE), a modified PTFE (TFM), a fluorinated ethylene propylene (FEP), a polyvinylidene fluoride (PVDF), a perfluoroalkoxy (PFA), a terpolymer of tetrafluoroethylene, a hexafluoropropylene and vinylidene fluoride (THV), a polychlorotrifluoroethylene (PCTFE), an ethylene tetrafluoroethylene copolymer (ETFE), an ethylene chlorotrifluoroethylene copolymer (ECTFE), or any combination thereof. Other fluoropolymers, polymers, and blends may be included in the composition of the apparatus 100 or any components thereof listed herein. In another particular embodiment, the apparatus 100 or any components thereof listed herein can at least partially include, or even consist essentially of, a polyethylene (PE) such as an ultra-high-molecular-weight polyethylene (UHMWPE). In another particular embodiment, the apparatus 100 or any components thereof listed herein may include a thermoplastic elastomeric hydrocarbon block copolymer, a polyether-ester block co-polymer, a thermoplastic polyamide elastomer, a thermoplastic polyurethane elastomer, a thermoplastic polyolefin elastomer, a thermoplastic vulcanizate, an olefin-based co-polymer, an olefin-based ter-polymer, a polyolefin plastomer, or combinations thereof. In an embodiment, the apparatus 100 or any components thereof listed herein may include a styrene based block copolymer such as styrene-butadiene, styrene-isoprene, blends or mixtures thereof, and the like. Exemplary styrenic thermoplastic elastomers include triblock styrenic block copolymers (SBC) such as styrene-butadiene-styrene (SBS), styrene-isoprene-styrene (SIS), styrene-ethylene butylene-styrene (SEBS), styrene-ethylene propylene-styrene (SEPS), styrene-ethylene-ethylene-butadiene-styrene (SEEBS), styrene-ethylene-ethylene-propylene-styrene (SEEPS), styrene-isoprene-butadiene-styrene (SIBS), or combinations thereof. Commercial examples include some grades of Kraton™ and Hybrar™ resins. In an embodiment, the apparatus 100 or any components thereof listed herein may include an elastomer including at least one of Acrylonitrile-Butadiene (NBR) Carboxylated Nitrile (XNBR) Ethylene Acrylate (AEM, Vamac®), Ethylene Propylene Rubber (EPR, EPDM), Butyl Rubber (IIR), Chloroprene Rubber (CR), Fluorocarbon (FKM, FPM), Fluorosilicone (FVMQ), Hydrogenated Nitrile (HNBR), Perfluoroelastomer (FFKM), Polyacrylate (ACM), Polyurethane (AU, EU), Silicone Rubber (Q, MQ, VMQ, PVMQ), Tetrafluoroethylene-Propylene (AFLAS®) (FEPM). In a number of embodiments, the apparatus 100 or any components thereof listed herein may be formed from any conventional methods known for polymer manufacturing, including but not limited to injection molding, billeting, cutting/slicing, hot or cold pressing, direct forming, extrusion, skiving, machining, blow molding, overmolding, vacuum forming, or CNC machining.

In an embodiment, the apparatus 100 or any components thereof listed herein (including, but not limited to, the core 110, the at least one retainer 120, the at least one membrane 130, or the ring seal 160) can at least partially include a rigid material such as, but not limited to, a metal. According to certain embodiments, the metal may include iron, copper, titanium, tin, aluminum, alloys thereof, or may be another type of metal. In an embodiment, the apparatus 100 or any components thereof listed herein can include a metal (such as aluminum, zinc, copper, magnesium, tin, platinum, titanium, tungsten, iron, bronze, steel, energizer steel, stainless steel), a metal alloy (including the metals listed), an anodized metal (including the metals listed), or any combination thereof. In a number of embodiments, the apparatus 100 or any components thereof listed herein may be formed from any conventional methods known for metalworking, such as turning, forging, extruding, molding, micromolding, micromachining, sintering, rolling, casting, injection molding, metalworking, flattening, hydroforming, punching, CNC machining, or 3-D printing (including binderjetting, stereolithography, filament disposition method, laser melting, laser sintering, electron beam melting).

In an embodiment, the apparatus 100 or any components thereof listed herein (including, but not limited to, the core 110, the at least one retainer 120, the at least one membrane 130, or the ring seal 160) can include a ceramic such as alumina, silica, titanium dioxide, calcium fluoride, boron nitride, mica, wollastonite, glass fibers, silicon carbide, silicon nitride, zirconia, carbon black, pigments, or any combination thereof. In a number of embodiments, the apparatus 100 or any components thereof listed herein may be formed from any conventional methods known for ceramic manufacturing, such as turning, extruding, molding, micromolding, micromachining, sintering, casting, injection molding, flattening, hydroforming, punching, CNC machining, or 3-D printing (including binderjetting, stereolithography, filament disposition method, laser melting, laser sintering, electron beam melting).

In an embodiment, the apparatus 100 or any components thereof listed herein (including, but not limited to, the core 110, the at least one retainer 120, the at least one membrane 130, or the ring seal 160) can be treated, impregnated, filled, or coated with a lubricious material or filler as a coating or surface treatment. Exemplary lubricious materials or fillers include molybdenum disulfide, tungsten disulfide, graphite, grapheme, expanded graphite, boron nitride, talc, calcium fluoride, or any combination thereof. Additionally, the lubricious material or filler can include a ceramic such as alumina, silica, titanium dioxide, calcium fluoride, boron nitride, mica, wollastonite, glass fibers, silicon carbide, silicon nitride, zirconia, carbon black, pigments, or any combination thereof. In an embodiment, the apparatus 100 or any components thereof listed herein may be a material that is resistant to oxygen, hydrogen, hydroxide, or temperature resistant. In a number of embodiments, the apparatus 100 or any components thereof listed herein may be monolithic. In alternative embodiments, the apparatus 100 or any components thereof listed herein may not be monolithic and may include multiple pieces.

In an embodiment, the apparatus 100 or any components thereof listed herein (including, but not limited to, the core 110, the at least one retainer 120, the at least one membrane 130, or the ring seal 160) can advantageously withstand sterilization processes. In an embodiment, the apparatus 100 or any components thereof listed herein may be sterilized by any method envisioned. For instance, the polymer of the apparatus 100 is sterilized after the apparatus 100 is formed. Exemplary sterilization methods include radiation (such as X-ray radiation), electron ray, E-beam or electron beam sterilization techniques, combinations thereof, and the like. In a particular embodiment, the polymer or polymeric blend is sterilized by vaporized hydrogen peroxide sterilization (VHP). In a particular embodiment, the apparatus 100 or any components thereof listed herein is sterilized by gamma irradiation. For instance, the apparatus 100 or any components thereof listed herein may be gamma sterilized at between about 25 kGy to about 50 kGy.

In embodiment, the apparatus 100 or any components thereof listed herein (including, but not limited to, the core 110, the at least one retainer 120, the at least one membrane 130, or the ring seal 160) may have further desirable physical and mechanical properties. For instance, the apparatus 100 or any components thereof listed herein may appear transparent or at least translucent. For instance, the apparatus 100 or any components thereof listed herein may have a light transmission greater than about 2%, or greater than about 5% in the visible light wavelength range. In particular, the resulting articles have desirable clarity or translucency. In addition, the apparatus 100 or any components thereof listed herein may have advantageous physical properties, such as a balance of any one or more of the properties of hardness, flexibility, surface lubricity, tensile strength, elongation, Shore A hardness, gamma resistance, weld strength, and seal integrity to an optimum level.

In an embodiment, the apparatus 100 or any components thereof listed herein (including, but not limited to, the core 110, the at least one retainer 120, the at least one membrane 130, or the ring seal 160) may have desirable heat stability properties. Applications for the polymer or polymeric blend are numerous. In particular, the apparatus 100 or any components thereof listed herein may be non-toxic, making the material useful for any application where no toxicity is desired. For example, the apparatus 100 or any components thereof listed herein may be substantially free of plasticizers or other low-molecular weight extenders that can be leached into the fluids it transfers. “Substantially free” as used herein refers to a polymeric mixture having a total organic content (TOC) (measured in accordance to ISO 15705 and EPA 410.4) of less than about 100 ppm. Further, the apparatus 100 or any components thereof listed herein may have biocompatibility and animal derived component-free formulation ingredients. For instance, the apparatus 100 or any components thereof listed herein may have potential for FDA, USP, EP, ISO, and other regulatory approvals. In an exemplary embodiment, the apparatus 100 or any components thereof listed herein may be used in applications such as industrial, medical, health care, biopharmaceutical, pharmaceutical, drinking water, food & beverage, laboratory, dairy, and the like. In an embodiment, the apparatus 100 or any components thereof listed herein may be used in applications where low temperature resistance is desired. In an embodiment, the apparatus 100 or any components thereof listed herein may also be safely disposed as it generates substantially no toxic gases when incinerated and leaches no plasticizers into the environment if land filled.

In some embodiments, as shown best in FIG. 1D, the apparatus 100 may be configured to contain and modify and/or sustain a product 170 within its internal void 150 to form a system. The product 170 may include medium containing a medical, biological, or pharmaceutical component. In a number of embodiments, the medical, biological, or pharmaceutical component may at least one of a cell, biofluid, virus, microphage, protein, antibody, pharmaceutical, therapeutic, buffer, or combinations thereof. In a number of embodiments, the product 170 may include a fluid for use in medical, biological, or pharmaceutical applications. In a number of embodiments, the product 170 may include a biomedia fluid for use in medical, biological, or pharmaceutical applications such as, but not limited to, cell cultures. In a number of embodiments, the medically, biologically, or pharmaceutically active environment within the apparatus 100 according to embodiments herein may be adapted to undergo a biological process or chemical reaction to modify or sustain the medical, biological, or pharmaceutical component 170 within the medium as provided by the medically, biologically, or pharmaceutically active environment within the internal void 150 of the apparatus 100.

In accordance with embodiments described herein, a method of making a system for storing medical, pharmaceutical, or biological media is shown. The method can include providing a medium including a medical, pharmaceutical, or biological component 170. The method can further include providing an apparatus 100 including: a core 110 including an annular body 112 defining an internal void 150 oriented down a central axis 1000 having a top axial surface 112A and a bottom axial surface 112B, at least one retainer 120 including an annular body 122 adapted to couple to the top axial surface 122A or the bottom axial surface 112B of the core 100 to form a circumferential engagement cavity 140 defined radially between the at least one retainer 120 and the core 110, at least one ring seal 160 disposed within the engagement cavity 140, and at least one flexible film membrane 130 partially disposed within the engagement cavity 140, where the at least one flexible film membrane 130 at least partially encloses the internal void 150. The method may further include disposing the medium 170 within the internal void 150 to provide for a medically, biologically, or pharmaceutically active environment within the internal void 150 of the core 110 and subsequent apparatus 100.

In a particular instance, apparatuses, systems and methods described herein can be adapted for use with medical, biological, or pharmaceutical media. In an embodiment, the apparatuses, systems and methods described herein can be used to prevent wrinkling, enabling for more efficient storage, better gas diffusion, better handling, and better operation involving modification or sustainment of the media within the apparatus unlike previous or conventional apparatuses of similar use. This may lengthen the lifetime and efficiency of the apparatus and the medical, biological, or pharmaceutical media cultivated therein.

Many different aspects and embodiments are possible. Some of those aspects and embodiments are described below. After reading this specification, skilled artisans will appreciate that those aspects and embodiments are only illustrative and do not limit the scope of the present invention. Embodiments may be in accordance with any one or more of the items as listed below.

Embodiment 1: An apparatus comprising: a core comprising an annular body defining an internal void oriented down a central axis having a top axial surface and a bottom axial surface; at least one retainer comprising an annular body adapted to couple to the top axial surface or the bottom axial surface of the core to form a circumferential engagement cavity defined radially between the at least one retainer and the core; at least one ring seal disposed within the engagement cavity; and at least one flexible film membrane partially disposed within the engagement cavity, wherein the at least one flexible film membrane at least partially encloses the internal void to provide for a medically, biologically, or pharmaceutically active environment within the internal void of the core.

Embodiment 2: A system comprising: a medium comprising a medical, pharmaceutical, or biological component; and an apparatus comprising: a core comprising an annular body defining an internal void oriented down a central axis having a top axial surface and a bottom axial surface, at least one retainer comprising an annular body adapted to couple to the top axial surface or the bottom axial surface of the core to form a circumferential engagement cavity defined radially between the at least one retainer and the core, at least one ring seal disposed within the engagement cavity, and at least one flexible film membrane partially disposed within the engagement cavity, wherein the at least one flexible film membrane at least partially encloses the internal void about the medium to provide for a medically, biologically, or pharmaceutically active environment within the internal void of the core.

Embodiment 3: A method comprising: providing a medium comprising a medical, pharmaceutical, or biological component; providing an apparatus comprising: a core comprising an annular body defining an internal void oriented down a central axis having a top axial surface and a bottom axial surface, at least one retainer comprising an annular body adapted to couple to the top axial surface or the bottom axial surface of the core to form a circumferential engagement cavity defined radially between the at least one retainer and the core, at least one ring seal disposed within the engagement cavity, and at least one flexible film membrane partially disposed within the engagement cavity, wherein the at least one flexible film membrane at least partially encloses the internal void; and disposing the medium within the internal void to provide for a medically, biologically, or pharmaceutically active environment within the internal void of the core.

Embodiment 4: The apparatus, system, or method of any of the preceding embodiments, wherein the at least one retainer comprises a plurality of retainers, wherein a first retainer couples to the top axial surface of the core to form a first engagement cavity and a second retainer couples to a bottom axial surface of the core to form a second engagement cavity.

Embodiment 5: The apparatus, system, or method of embodiment 4, wherein the at least one flexible film membrane comprises a plurality of flexible film membranes.

Embodiment 6: The apparatus, system, or method of embodiment 5, wherein the plurality of flexible film membranes comprises a first flexible film membrane partially disposed within the first engagement cavity and a second flexible film membrane partially disposed within the second engagement cavity.

Embodiment 7: The apparatus, system, or method of any of the preceding embodiments, wherein the at least one flexible film membrane comprises a polymer that is gas-permeable and liquid-impermeable.

Embodiment 8: The apparatus, system, or method of embodiment 7, wherein the polymer comprises EVA, silicone, polyolefin, polycarbonate, HDPE, POE, COC, COP, PMP, FEP, PTFE, or a combination thereof.

Embodiment 9: The apparatus, system, or method of any of the preceding embodiments, wherein the at least one flexible film membrane comprises a monolayer.

Embodiment 10: The apparatus, system, or method of any of the preceding embodiments, wherein the at least one flexible film membrane comprises a multilayer laminate.

Embodiment 11: The apparatus, system, or method of any of the preceding embodiments, wherein the at least one flexible film membrane has a thickness of greater than 0.001 inches.

Embodiment 12: The apparatus, system, or method of any of the preceding embodiments, wherein the at least one flexible film membrane is tensioned in a uniform pattern to at least partially enclose the internal void by contacting the core, the at least one retainer, and the at least one ring seal.

Embodiment 13: The apparatus, system, or method of any of the preceding embodiments, wherein the at least one flexible film membrane is tensioned such that it has a flatness having a deviation of no greater than 3 mm against a line perpendicular to a central axis.

Embodiment 14: The apparatus, system, or method of any of the preceding embodiments, wherein the core comprises at least one axially oriented coupling component to couple with the core comprising at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, bores, welds, or combinations thereof.

Embodiment 15: The apparatus, system, or method of any of the preceding embodiments, wherein the at least one retainer comprises at least one axially oriented coupling component to couple with the core comprising at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, bores, welds, or combinations thereof.

Embodiment 16: The apparatus, system, or method of embodiment 15, wherein the at least one coupling component of the at least one retainer couples to a neighboring retainer of a neighboring apparatus.

Embodiment 17: The apparatus, system, or method of embodiment 15, wherein the coupling component of the core and the coupling component of the at least one retainer snap-fit together to tension the at least one flexible film membrane.

Embodiment 18: The apparatus, system, or method of any of the preceding embodiments, wherein the annular body of the core comprises at least one access port disposed on the core, wherein the at least one access port allows for entrance or exit of a medium into or out of the apparatus.

Embodiment 19: The apparatus, system, or method of embodiment 4, wherein the at least one ring seal comprises a plurality of ring seals.

Embodiment 20: The apparatus, system, or method of embodiment 19, wherein the plurality of ring seals comprises a first ring seal disposed within the first engagement cavity and a second ring seal disposed within the second engagement cavity.

Embodiment 21: The apparatus, system, or method of any of the preceding embodiments, wherein the at least one ring seal comprises an elastomer.

Embodiment 22: The apparatus, system, or method of any of the preceding embodiments, wherein the at least one of the retainer comprises a rigid material comprising a ceramic.

Embodiment 23: The apparatus, system, or method of any of the preceding embodiments, wherein the at least one of the retainer comprises a rigid material comprising a polymer.

Embodiment 24: The apparatus, system, or method of any of the preceding embodiments, wherein the at least one of the retainer comprises a rigid material comprising a metal.

Embodiment 25: The apparatus, system, or method of any of the preceding embodiments, wherein the core comprises a rigid material comprising a ceramic.

Embodiment 26: The apparatus, system, or method of any of the preceding embodiments, wherein the core comprises a rigid material comprising a polymer.

Embodiment 27: The apparatus, system, or method of any of the preceding embodiments, wherein the core comprises a rigid material comprising a metal.

Embodiment 28: The apparatus, system, or method of any of the preceding embodiments, wherein the core has a substantially U-shaped cross-sectional profile in a plane parallel to the central axis.

Embodiment 29: The apparatus, system, or method of any of the preceding embodiments, wherein the at least one retainer has a modified L-shaped cross-sectional profile in a plane parallel to the central axis.

Embodiment 30: The apparatus, system, or method of any of the preceding embodiments, wherein the medically, biologically, or pharmaceutically active environment allows for a biological process to take place to modify or sustain the medium.

Embodiment 31: The apparatus, system, or method of any of the preceding embodiments, wherein the medical, pharmaceutical, or biological component comprises at least one of a cell, biofluid, virus, microphage, protein, antibody, pharmaceutical, therapeutic, or combinations thereof.

Embodiment 32: The apparatus, system, or method of any of the preceding embodiments, wherein the apparatus comprises a surface treatment or coating comprising a lubricious metal or a filler.

Note that not all of the activities described above in the general description or the examples are required, that a portion of a specific activity may not be required, and that one or more further activities may be performed in addition to those described. Still further, the order in which activities are listed is not necessarily the order in which they are performed.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

What is claimed is:

1. An apparatus comprising:
a core comprising an annular body defining an internal void oriented down a central axis having a top axial surface and a bottom axial surface;
at least one retainer comprising an annular body adapted to couple to the top axial surface or the bottom axial surface of the core to form a circumferential engagement cavity defined radially between the at least one retainer and the core;
at least one ring seal disposed within the engagement cavity; and
at least one flexible film membrane partially disposed within the engagement cavity,
wherein the at least one flexible film membrane at least partially encloses the internal void to provide for a medically, biologically, or pharmaceutically active environment within the internal void of the core.

2. A system comprising:
a medium comprising a medical, pharmaceutical, or biological component; and
an apparatus comprising:
a core comprising an annular body defining an internal void oriented down a central axis having a top axial surface and a bottom axial surface,
at least one retainer comprising an annular body adapted to couple to the top axial surface or the bottom axial surface of the core to form a circumferential engagement cavity defined radially between the at least one retainer and the core,
at least one ring seal disposed within the engagement cavity, and
at least one flexible film membrane partially disposed within the engagement cavity, wherein the at least one flexible film membrane at least partially encloses the internal void about the medium to provide for a medically, biologically, or pharmaceutically active environment within the internal void of the core.

3. A method comprising:
providing a medium comprising a medical, pharmaceutical, or biological component;
providing an apparatus comprising:
a core comprising an annular body defining an internal void oriented down a central axis having a top axial surface and a bottom axial surface,
at least one retainer comprising an annular body adapted to couple to the top axial surface or the bottom axial surface of the core to form a circumferential engagement cavity defined radially between the at least one retainer and the core,
at least one ring seal disposed within the engagement cavity, and
at least one flexible film membrane partially disposed within the engagement cavity, wherein the at least one flexible film membrane at least partially encloses the internal void; and
disposing the medium within the internal void to provide for a medically, biologically, or
pharmaceutically active environment within the internal void of the core.

4. The apparatus of claim 1, wherein the at least one retainer comprises a plurality of retainers, wherein a first retainer couples to the top axial surface of the core to form a first engagement cavity and a second retainer couples to a bottom axial surface of the core to form a second engagement cavity.

5. The apparatus of claim 4, wherein the at least one flexible film membrane comprises a plurality of flexible film membranes.

6. The apparatus of claim 5, wherein the plurality of flexible film membranes comprises a first flexible film membrane partially disposed within the first engagement cavity and a second flexible film membrane partially disposed within the second engagement cavity.

7. The apparatus of claim 1, wherein the at least one flexible film membrane comprises a polymer that is gas-permeable and liquid-impermeable.

8. The apparatus of claim 1, wherein the at least one flexible film membrane comprises a monolayer.

9. The apparatus of claim 1, wherein the at least one flexible film membrane comprises a multilayer laminate.

10. The apparatus of claim 1, wherein the at least one flexible film membrane is tensioned in a uniform pattern to at least partially enclose the internal void by contacting the core, the at least one retainer, and the at least one ring seal.

11. The apparatus of claim 1, wherein the at least one flexible film membrane is tensioned such that it has a flatness having a deviation of no greater than 3 mm against a line perpendicular to a central axis.

12. The apparatus of claim 1, wherein the core comprises at least one axially oriented coupling component to couple with the core comprising at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, bores, welds, or combinations thereof.

13. The apparatus of claim 1, wherein the at least one retainer comprises at least one axially oriented coupling component to couple with the core comprising at least one of nuts, bolts, bearings, battens, buckles, clips, flanges, frogs, grommets, hook-and-eyes, latches, pegs, nails, rivets, tongue and grooves, screw anchors, snap fasteners, stitches, threaded fasteners, ties, toggle bolts, wedges anchors, screws, bolts, clamps, clasps, clips, latches, pins, rivets, ties, nails, bores, welds, or combinations thereof.

14. The apparatus of claim 13, wherein the at least one coupling component of the at least one retainer couples to a neighboring retainer of a neighboring apparatus.

15. The apparatus of claim 13, wherein the coupling component of the core and the coupling component of the at least one retainer snap-fit together to tension the at least one flexible film membrane.

16. The apparatus of claim 1, wherein the annular body of the core comprises at least one access port disposed on the core, wherein the at least one access port allows for entrance or exit of a medium into or out of the apparatus.

17. The apparatus of claim 16, wherein the plurality of ring seals comprises a first ring seal disposed within the first engagement cavity and a second ring seal disposed within the second engagement cavity.

18. The apparatus of claim 1, wherein the core has a substantially U-shaped cross-sectional profile in a plane parallel to the central axis.

19. The apparatus of claim 1, wherein the at least one retainer has a modified L-shaped cross-sectional profile in a plane parallel to the central axis.

20. The system of claim 2, wherein the medical, pharmaceutical, or biological component comprises at least one of a cell, biofluid, virus, microphage, protein, antibody, pharmaceutical, therapeutic, or combinations thereof.
